(19) United States
(12) Patent Application Publication
(10) Pub. No.: US 2026/0124431 A1
(43) Pub. Date: May 7, 2026

(54) STEERABLE DEVICE FOR USE INSIDE OF A MAMMALIAN BODY

(71) Applicant: ETH ZÜRICH, Zürich (CH)

(72) Inventors: Sean Liam LYTTLE, Uster (CH); Roland DREYFUS, Zürich (CH); Alexandre MESOT, Zürich (CH); Bradley NELSON, Zumikon (CH)

(73) Assignee: ETH ZÜRICH, Zürich (CH)

(21) Appl. No.: 19/118,191

(22) PCT Filed: Oct. 5, 2023
(86) PCT No.: PCT/EP2023/077631

§ 371 Date: Apr. 3, 2025

(30) Foreign Application Priority Data
Oct. 7, 2022 (EP) 22200349.3

(51) Int. Cl. A61M 25/01; A61B 1/00; A61B 1/005; A61B 1/008; A61M 25/00; A61M 25/06

(52) U.S. Cl. CPC A61M 25/0127; A61M 25/005; A61M 25/0068; A61M 25/0662; A61B 1/00128; A61B 1/00158; A61B 1/0055; A61B 1/008

(57) ABSTRACT

A steerable device includes an elongated element configured to be navigated through the mammalian body. The elongated element has a device tip configured for allowing steering of the elongated element by way of an external magnetic field. Further, the device tip includes a plurality of segments, each segment including a segment body and a segment interface, and a plurality of connectors, each connector including a connector body and a connector interface. The connector interfaces and the segment interfaces have each time mutually complementing shapes allowing rotational movements relative to each other and forming a connection to retain the connectors and the segments in a mated condition, and the magnetic elements form at least partially segment bodies or connector bodies or both.

The invention relates to a steerable device for use inside of a mammalian body. The device may be a needle, guidewire, catheter, endoscope, or any other flexible device designed to be inserted into a mammalian body, especially lumens or cavities, for diagnostic or interventional purposes.

Many medical procedures require a medical instrument to be navigated to a specific location inside the body. Examples include deep brain stimulation, organ biopsies, targeted drug delivery, tumor removal, and many others. The insertion and navigation of such devices is achieved in a variety of ways depending on the application.

In the case of procedures inside body cavities, such as the abdomen or inside the bladder, or inside a vasculature system a flexible device is steered, usually by means of pull wires or rotating a pre-curved distal tip, while being pushed proximally. There is an inherent tradeoff between the device's ability to reach target sites and its ability to be advanced without buckling.

Some proposed devices have a magnetic tip that allows a device to be more flexible and its trajectory to be more precisely controlled by way of a magnetic field. However, magnetic steering generally requires a tradeoff between magnetic material volume present at the magnetic tip and stiffness, in particular axial stiffness. Because torques generated by magnetic steering are usually lower than torques generated by traditional pull wire steering, magnetically steered devices must have a magnetic tip which is very flexible. Further, the magnetic tip must have also a sufficient volume of magnetic material to be effectively steered. Normally, these two objectives are at odds because magnetic material is rigid and compromises the flexibility of the device tip. Additionally, the flexibility required by magnetically steered devices prevents more rigid tools like biopsy probes to be used in conjunction with the device and the volume of magnetic material needed is a parameter limiting the diameter reduction of the device.

Traditionally, magnetically steered medical devices comprise one or more magnets separated by a very flexible polymer.

U.S. Pat. No. 3,674,014 discloses a flexible catheter-tip, guidable by a magnetic field into selected arteries of the body, including a plurality of permanent magnetic tubular segments with ball-shaped ends arranged end-to-end. Each pair of adjacent ends is encased within a tubular link, i.e. a non complementing shape with regard to the corresponding ball-shaped end, formed of non-magnetic material which provides a flexible, fluid-tight seal between the tubular segments. Each ball-shaped end has formed thereon a bevel to provide stability between adjacent segments, and therefore to the whole catheter-tip, at full bend. The diameter of such a device is limited by the fact that the diameter of the tubular segments with ball-shaped ends and of the tubular links are limited by their mechanical resistance.

There remains a need to reach target sites that are situated even further in body cavities or vasculature systems through acute bends at branch junctions in the body passages and lumens having smaller dimension with increasing distances. At the same time, there is also a need to navigate the medical device in a stable manner without kinking in open spaces, such as the intestine, bladder, stomach or other body cavities.

It is an object of the present invention to provide a device for use inside of a mammalian body, which can be used in a reliable and reproducible manner in applications for diagnostic and/or interventional purposes requiring a stable navigation in open spaces without kinking and a great precision in the navigation to reach target sites through acute bends at branch junctions in the body passages and through lumens having smaller dimensions, and which is not subject to the limitations of the devices listed above.

This objective is met according to the present invention by providing a device with the features of claims 1 and 15. Further advantageous embodiments of the invention are the subject of the dependent claims.

A steerable device is disclosed for use inside of a mammalian body, the steerable device being a needle, guidewire, catheter, endoscope, or the like that is used inside of a mammalian body, in particular of a human, for example to inspect and/or operate. The steerable device may be used for inspecting and/or operating inside of organs (e.g. stomach, intestines, liver, lungs, kidney, brain, etc.), inside of a body cavity (e.g. abdomen, spinal cord, sinuses etc.), or in the vasculature.

The steerable device comprises an elongated element that is flexible and elongated along a device longitudinal axis, wherein the elongated element is flexible and configured to enter soft tissues of the mammalian body through an incision or orifice or other suitable means and to be navigated through the mammalian body.

Further, the elongated element comprises a device tip which is flexible and configured for allowing steering of the elongated element by way of an external magnetic field. The device tip is preferably arranged at a distal end of the elongated element. The device tip is configured to be bent by way of an external magnetic field to allow steering of the elongated element.

An end of the steerable device opposed to the device tip, i.e. a proximal end of the steerable device, is designed to remain outside of the mammalian body for external manipulation for example by a physician or a robot.

In the context of the present disclosure, distal refers to a position or a direction that is on the side of an operation site, i.e. on a patient side, and proximal refers to a position or a direction that is on the opposed side, i.e. the side that is farther from the patient side and where an operator can be.

In particular, the elongated element ends distally in the device tip. The distal tip may contain or carry measurement and/or actuating devices, such as a camera, sensors, ablation tip, needle, electrodes, etc. The device may contain a lumen that allows delivery of fluids, needles, coils, drugs, biopsy tools and the like to a target site.

The device tip comprises a plurality of segments, each segment including a segment body and a segment interface arranged at each longitudinal end of the segment body, i.e. a segment has each time two segment interfaces, namely a distal and a proximal segment interface.

Additionally, the device tip comprises a plurality of connectors, each connector including a connector body and a connector interface arranged at each longitudinal end of the connector body, i.e. a connector has each time two connector interfaces, namely a distal and a proximal connector interface.

The plurality of segments and the plurality of connectors are arranged along the device longitudinal axis, each connector connecting two consecutive segments in a manner allowing movements of the segments and the connectors relative to each other in an assembled state thereof to allow bending of the device tip.

The plurality of segments and the plurality of connectors can be arranged in a tubular section of the device tip designed to limit radial movements of the segments and the connectors from the device longitudinal axis. The tubular section can be in the form of a tube enveloping the plurality of connectors and the plurality of segments, the tube being attached to the distal end of the elongated element or formed as a hollow extension of the elongated element, more specifically of an external envelope of the elongated element, in the longitudinal direction. In particular, the tubular section can be a part of the envelope. The tubular section keeps the plurality of connectors and the plurality of segments aligned or at least approximately aligned in the device longitudinal direction.

According to the invention, the connector interfaces and the segment interfaces have each time mutually complementing shapes allowing rotational movements relative to each other and forming a connection to retain the connectors and the segments aligned along the longitudinal axis in a mated condition, Further, according to the invention, magnetic elements form at least partially segment bodies or connector bodies or both.

Mutually complementing shapes refer to shapes acting as a complement to each other and fit another exactly or with just enough clearance to allow a relative motion between each other. Mutually completing parts can be for example counterparts that fit another exactly, like a ball and a socket in a ball-joint connection. On the contrary, a ball-shaped part and a cylindrical tube, the ball-shaped part being encased in the cylindrical tube having an internal diameter corresponding to the diameter of the ball, do not form mutually complementing shapes.

The connector interfaces and the segment interfaces having a mutually complementing shape improve the stability of the device tip in particular in the longitudinal direction. The mutually complementing shape limits slipping and improves force transmission at the interface between connectors and segments because they have an increased surface of contact. At the same time bending of the device tip is not unduly limited because the connector interfaces and the segment interfaces are designed to allow rotational movement of connectors and segments relative to each.

Further, the thickness of the tubular section can be reduced because a part of its function of keeping the segments and connectors aligned is transferred to the connector interfaces and the segment interfaces. Accordingly, the diameter of the device can be reduced. Alternatively, the thickness of the tubular section and the diameter of the device can be kept constant and, in this case, the longitudinal alignment of the segments and connectors in the device tip is improved.

Magnetic materials are brittle and have limited resistance to mechanical constraints present at the interfaces between segments and connectors. Therefore, it is an advantage when magnetic elements are formed as or form a part of connector bodies, segment bodies or both, where significantly lower mechanical constraints are present. It follows that the device tip has an improved force transmission at the interface between connectors and segments which is not limited by mechanical properties of magnetic material. As a result, there is no need to provide for solid magnetic elements designed to resist the mechanical constraints at the interfaces. This design also allows a reduction of diameter of the steerable device. At the same time, magnetic material can be distributed over the length of the device tip in the form of magnetic elements associated to the segments and connectors either as forming the segments and connectors or forming a part of the segments and connectors.

In addition, the volume of magnetic material can be adjusted by designing the device tip to include magnetic elements distributed over segment bodies, connector bodies or both. This feature advantageously allows the design of steerable devices that can be effectively steered in magnetic fields.

In a preferred embodiment, magnetic elements are associated to each segment and connector to maximize the volume of magnetic material.

In a preferred embodiment, the connection is a ball-joint connection comprising each time a ball and a socket arranged to connect a segment interface to an adjacent connector interface in the direction of the device longitudinal axis, an inclination angle α being formed each time between a segment longitudinal axis of a segment and a connector longitudinal axis of a connector adjacent to the segment when the device tip is bent, wherein the inclination angle is measured in a plane formed by the segment longitudinal axis and the connector longitudinal axis. The ball-joint connections form a plurality of ball-joint connections. The balls and the sockets together form each time interlocking components of a snap-fit assembly forming the ball-joint connections. This method of connection avoids the risk k of slipping and further s force transmission at the interface between connectors and segments without compromising bending of the device tip. Further, it is a particularly efficient connection between segments and connectors to avoid kinking of the steerable device so that a navigation in a stable manner in open spaces is possible.

In a preferred embodiment, the ball-joint connection is formed in a manner such that the segment interfaces comprise each time the ball and the connector interfaces comprise each time the socket designed to receive the ball.

More preferably the segment interfaces are formed each time by the ball and the connector interfaces are formed each time by the socket to create a particularly compact assembly of the segments and connectors allowing more acute bending of the device tip.

It must also be noted that embodiments are also possible in which the segment interfaces comprise each time the socket and the connector interfaces comprise each time the ball. In the same way, it is also conceivable to have embodiments in which segments have each time a first segment interface comprising the ball and a second segment interface, that is arranged at the end of the segment opposed to the first segment interface, comprising the socket. In this embodiment, the connectors are formed in the same way, i.e. each connector has a ball and a socket at opposed ends of the connector. In a particularly preferred embodiment, the connectors and the segments have the same form and are interchangeable, which simplifies the production of the device tip.

Preferably, the segments and the connectors have an axial symmetry to provide for a simplified production.

The sockets have each time a socket wall that is delimited by a socket outer surface and a socket inner surface. The balls have each time a ball outer surface and can be solid. In case balls are hollow, the balls have each time a ball wall that is delimited by the ball outer surface and a ball inner surface.

In a preferred embodiment, each ball-joint connection is formed such that the ball outer surface is spherical and the socket inner surface is spherical, wherein the ball extends axially further, as seen in the direction from the ball to the socket, than an equatorial plane of the socket, the socket having an opening with an opening diameter that is less than a ball diameter of the ball, an edge region of the socket comprising the opening forming a detent provided to retain the ball and the socket in a mated condition. The opening of the socket lies preferably in a plane that is parallel to the equatorial plane of the socket and has a circular form. Other embodiments of ball-joint connection may be used. A spherical ball has the advantage that the ball-joint connection can rotate freely in space. It is noted that the term “spherical” covers also shapes that are almost approximatively spherical within production tolerances.

It is also conceivable to use balls having an ovoid form or even a cylindrical shape in the ball-joint connections, if it is necessary to prevent bending in certain axes.

Preferably, the sockets have at least one cut extending axially from the opening in the direction opposed to the opening. The cuts allow an easier insertion of the balls into the sockets to simplify the assembly the device tip.

In a preferred embodiment, each segment and each connector have a bore extending in the direction of the device longitudinal axis and forming a plurality of bores, the plurality of bores forming a conduit extending axially through the plurality of segments, i.e. through the segment bodies and the segment interfaces, and through the plurality of connectors, i.e. through the connector bodies and the connector interfaces, in the assembled state. This conduit can be used to provide access for small tools and the like to the target site or to accommodate a tube to form a lumen that allows delivery for example of fluids to the target site.

In preferred embodiments, the magnetic elements each time surround at least partially the segment bodies or the connector bodies or both. Preferably, the magnetic elements are axially symmetric in the form of a cylinder centered on the device longitudinal axis to simplify steering in the magnetic field. For example, if the volume of magnetic material must be increased, the magnetic elements can run along the whole axial length of the segment bodies and connector bodies, respectively.

In other preferred embodiments, the magnetic elements can be at least partially embedded in the segment bodies or in the connector bodies or in both. Advantageously, the embedded magnetic elements contribute to the reduction of the diameter of the steerable device.

In a preferred embodiment, the magnetic elements are formed of permanent magnetic material to allow magnetic steering of the device tip.

In a preferred embodiment, the portion of the segments and of the connectors that do not form the magnetic elements are made of magnetically non-responsive material to avoid interaction with the magnetic field applied to steer the magnetic elements. This embodiment can be used when only a low volume of magnetic material is required to allow navigation of the steerable device.

In a preferred embodiment, the portion of the segments and of the connectors that do not form the magnetic elements are made of a ferrous material, for example a ferrous steel, which is magnetized by the permanent magnets and thereby contributes to the overall volume of magnetic material. The resulting increased interaction with the magnetic field applied to steer the magnetic element allows an improved steering.

A magnetically non-responsive material can be a non-ferromagnetic material to avoid interactions with the magnetic field applied to steer the steerable device. However, it can also be a material having diamagnetic or paramagnetic properties which magnetic contribution is negligible compared to the magnetic contribution of the magnetic elements to the steering of the device. These properties are such that the interaction of the segments and the connectors with the magnetic field is orders of magnitudes smaller than the interaction of the magnetic elements with the magnetic field and, consequently, can be neglected while steering the steerable device in the mammalian body.

The segments and connectors can be made of a material suitable for biomedical application like a polymer material such as nylon, polyurethane, polyethylene, polyether block amide (for example known under the tradename Pebax®), POM, PEEK or silicone. Alternatively, they can be made of a metal, for example steel, stainless steel, titanium, aluminum, or any other biocompatible metal. For the magnetic elements, materials like ferrite, neodymium iron boron (NdFeB), aluminum nickel cobalt (AlNiCo), samarium cobalt (SmCo), iron chromium cobalt (FeCrCo), or any other suitable ferromagnetic material or alloy can be used.

In a preferred embodiment, the plurality of connectors or the plurality of segments or both are made up of one piece. The simple design allows simplifies assembly and reduces production costs.

In a preferred embodiment, the connectors are made up of individual components in the form of the connector body, preferably formed as a tube element, and of the connector interfaces, the latter being inserted in the connector body at each end of the connector body.

In a further preferred embodiment, the segments are made up of individual components in the form of the segment body, preferably formed as a further tube element, and of the segment interfaces, the latter being inserted in the segment body at each end of the segment body. The individual components can be kept assembled by forces exerted along the device longitudinal direction at both end of the tubular section containing the plurality of segments and the plurality of connectors. The individual components can also be kept assembled by mechanical means including glue or welding. For an easy insertion in the segment bodies and in the connection bodies, the segment interfaces and the connection interfaces, respectively, can have an insertion portion for guiding the insertion, the insertion portion being arranged on their side facing the segment body and the connection body, respectively.

In another preferred embodiment, both the connectors and the segments are made up of individual components.

Advantageously, starting from individual components allows different configurations of the connectors and/or the segments without significantly increasing the complexity of the device tip production. For example, various lengths of segments and connectors can be achieved by using segment bodies and connector bodies, respectively, having different length while keeping the same segment interfaces and connector interfaces, respectively. This also allows the assembly of segments and connectors comprising different material for the segment bodies and the connector bodies, respectively. In particular, the segment bodies and the connector bodies are made of magnetic material and the segment interfaces and connector interfaces, respectively, are made of materials having different mechanical properties, e.g. materials allowing the formation of ball-joint connections.

In an even more preferred embodiment, the segment bodies and the connector bodies can have the same design so that they form interchangeable components, which in turns reduces the number of different components and simplifies the production of the device tip.

In a more preferred embodiment, the tube elements or the further tube elements or both form the magnetic elements. This preferred embodiment allows a further reduction of the diameter of the steerable device because the magnetic elements form at the same time the bodies of the segments and the connectors.

Magnetic elements can also be formed as half cylindrical shells or more generally portion of cylindrical shells that are assembled with the segment bodies and/or connector bodies.

In a preferred embodiment, the device tip comprises a first group of segments and connectors, preferably arranged distally, which segments and connectors have a first length and a second group of segments and connectors, preferably arranged proximally, which segments and connectors have a second length, as measured along the device longitudinal axis, the first length being shorter than the second length. This arrangement allows the formation of two regions in the device tip, namely a distal region of the device tip including the first group allowing acute bending, i.e. short curvature radius of the device, to facilitate the navigation of the distal region, and a proximal region of the device tip including the second group allowing less acute bending, i.e. larger curvature radius, and more axial stiffness.

It is also conceivable to provide for segments having different lengths and connectors having different lengths, wherein the lengths and the arrangement of the segments and the connectors are defined depending on the bending characteristics required for the steerable device.

In a preferred embodiment, the device tip comprises a group of segments and connectors, preferably arranged distally, which segments and connectors have magnetic elements and a further group of segments and connectors, preferably arranged proximally, which segments and connectors are free of magnetic elements. This arrangement allows also the formation of two regions in the device tip, namely a distal region of the device tip including the group with magnetic elements allowing navigation of the distal region under the effect of magnetic fields, and a proximal region of the device tip including the further group that is non responsive to magnetic fields but has more axial stiffness.

Advantageously, embodiments can be conceived that include a combination of the two previous preferred embodiments, namely a distal region including segments and connectors having a shorter length and having magnetic elements together with a proximal region including segments and connectors having longer length and being free of magnetic elements, to enhance the effects obtained and described previously.

In a preferred embodiment, the ball of ball-joint connections, preferably each ball of the plurality of ball-joint connections for simpler manufacturing, sits each time on a neck extending axially, and, for the segment and the connector of the ball-joint connections of the plurality of ball-joint connections, the inclination angle α is limited to a maximal inclination angle α_max reached when the neck is in a contact position with the edge region of the socket of the ball-joint connection as a result of a relative movement of the segment with respect to the connector, the edge region acting as a stopper for the inclination of the segment relative to the connector. The neck can be in the form of a cylinder protruding longitudinally from the segment body if the segment carries the ball in the ball-joint connection, or the connector body, if the connector carries the ball in the ball-joint connection, centered on the segment longitudinal axis or the connector longitudinal axis, respectively. The maximal inclination angle α_max can be predefined for an embodiment based in particular on the opening diameter of the socket and on a neck diameter of the neck. This arrangement avoids that the device tip is bent too acutely and consequently reduces the risk of a ball being pulled out of its corresponding socket, or a kink being formed in tubes or wires running through the inner passage. More preferably all ball-joint connections of the plurality of ball-joint connections are formed according to the embodiment just described to simplify the assembly and the production of the device tip.

In a preferred embodiment, the neck has a collar extending radially and arranged adjacent to the contact position on which the edge region of the socket abuts. Advantageously, the collar hinders further bending of the ball-joint connection by blocking the edge region at the contact position in the longitudinal direction. The provision of the collar extending radially from the ball is also possible.

In a preferred embodiment, the ball of ball-joint connections, preferably each ball of the plurality of ball-joint connections for simpler manufacturing, is truncated on the side of the ball facing the socket by a first plane, preferably parallel to an equatorial plane of the ball, forming a ball abutment surface and corresponding sockets of the plurality of ball-joint connections have a planar socket bottom, preferably parallel to an equatorial plane of the socket, on the side facing the ball, and, for the segment and the connector of the ball-joint connections of the plurality of ball-joint connections, the inclination angle α is limited to a maximal inclination angle α_max reached when the ball abutment surface is in a contact position with the socket bottom as a result of a relative movement of the segment with respect to the connector of the ball-joint connection, the contact position acting as a stopper for the inclination of the segment relative to the connector. The maximal inclination angle α_max can be predefined for an embodiment based in particular on the axial distance between the ball abutment surface and the socket position, as measured when the segment longitudinal axis and the connector longitudinal axis are parallel to each other. This arrangement also avoids that the device tip is bent too acutely and consequently reduces the risk of a ball being pulled out of its corresponding socket. More preferably all ball-joint connections of the plurality of ball-joint connections are formed according to the embodiment just described to simplify the assembly and the production of the device tip.

In a preferred embodiment, an outer surface of the sockets has a structured region and an interspace formed between the sockets and the tubular section in the assembled state of the device tip is filled with granular material, the tubular section surrounding at least the plurality of segments and the plurality of connectors, wherein granular material locks the relative position of the plurality of segments and of the plurality of connectors in a locked state when the interspace is under vacuum. For this purpose, the tubular section is connected to means to apply vacuum to the interspace.

The tubular section can be designed to form an airtight volume with a valve at the proximal end of the steerable device to allow a connection with a vacuum pump. Preferably, the tubular section is fitted closely to the surface of the segment bodies and the connector bodies and an interspace is formed by gaps at the ball-joint connection between the tubular section and segment bodies and connector bodies, in particular between the outer surface of the sockets of the ball-joint connection and the tubular section.

A structured region or structured area is to be understood as a surface having patterns that can be geometric features designed to give a specific function to the region. In the present case, the structured region increases friction between granular material and the sockets, for example by blocking granular material in depressions or between protrusions formed in the structured region. When the interspace is under vacuum, i.e. under pressures lower than atmospheric pressure, the envelope is pressed against the plurality of segments and the plurality of connectors as well as against granular material provided each time between segments and connectors. As a result, friction between granular material itself and between granular material and the sockets blocks the relative movement of the segments and of the connectors. For this purpose, the interspace is connected to means to apply vacuum.

Preferably, the structured surface has a structure in the form of a plurality of concentric grooves, spaced from each other, as seen in the longitudinal direction, each groove extending circumferentially in a plane parallel to the equatorial plane of the socket.

In a preferred embodiment, the segments and the connectors include each time at least three passages arranged in the respective bores forming the conduit at an equal peripheral distance from each other, the at least three passages forming each a through hole, wherein, in the assembled state, the through holes of the segments and the through holes of the connectors are aligned and the through holes form at least three guides through the plurality of segments and the plurality of connectors, the at least three guides being designed to allow each time the passage of a pull wire, wherein the pull wire is fixed in a distal region of the device tip and is designed to be pulled proximally to lock the relative position of the plurality of segments and the plurality of connectors. As a result of pulling proximally the pull wires, friction is increased at the ball-joint connection which locks the relative position of the segments and the connectors. The at least three passages can be arranged in the segments either in the segment bodies or in the segment interfaces and in the connectors either in the connector bodies or in the connector interfaces. At least three passages are necessary to block all degrees of freedom of the device tip. The at least three through holes of each segment and each connector form a plurality of through holes, wherein, in the assembled state, the plurality of through holes are aligned to form the at least three guides. Each guide can be seen as a path formed virtually by the alignment of the through holes of the plurality of through holes.

In other embodiments, the at least three passages can be in the form of lugs and are arranged in a plane perpendicular to the segment longitudinal axis and to the connector longitudinal axis, respectively, to allow for a symmetrical application of forces applied to the ball inner surface and pressing the ball outer surface on the socket inner surface.

Advantageously, the segment interface, in particular the ball or the ball neck, can also comprise a collar extending radially and arranged adjacent to the contact position on which the edge region of the connector interface, in particular the socket, abuts. It is also conceivable to form the collar as part of the segment body.

In a preferred embodiment, the segment bodies and the connector bodies comprise each time at least three passages on their distal side and at least three passages on their proximal side. As a result, each guide wire is supported in each segment and each connector by two passages that improves guiding of the guide wire and security in case one passage breaks.

In a preferred embodiment, a sleeve is arranged in the conduit, the sleeve comprising a braided sheath and a spring element surrounded by the braided sheath, wherein, in a relaxed state of the sleeve, the sleeve has an external diameter equal to or smaller than an internal diameter of the conduit and the spring element has an external diameter equal to or smaller than an inner diameter of the braided sheath, the sleeve extending from a distal sleeve end fixed in a distal region of the device tip to a proximal sleeve end designed to be pulled proximally to switch from the relaxed state to a stretched state of the sleeve in which the relative position of the plurality of segments and of the plurality of connectors is locked. To prevent the braided sheath from collapsing and/or shrinking in diameter while an operator pulls the sleeve, the spring element, which can be a wire spring, is designed to maintain the inner diameter of the braided sheath constant or at least approximately constant. Preferably, a distal end of the spring element is fixed in the distal region of the device tip, preferably at the same position as the distal sleeve end. For example, the distal sleeve end can be fixed at the most distal segment or as the case may be the most distal connector, of the arrangement formed by the plurality of segments and the plurality of connectors. The distal end of the spring element can be fixed at the same level. By pulling the braided sheath, braids of the braided sheath move relative to each and tend to align with the direction of pulling forces. As a result, the diameter of the braided sheath tends to become smaller and press against t an external surface of the spring element arranged inside the braided sheath, the spring element maintaining at least approximately the form of the braided sheath. While braids align along pulling forces, the sleeve presses multiple contacts within the segment interfaces, in particular within the balls, against the connector interfaces, in particular the sockets, thereby increasing friction at the ball-joint connections and locking segments and connectors relative to each other.

This arrangement can be used as an alternative to the use of pull wires previously described.

In a preferred embodiment, ball-joint connections have a socket comprising through bores that extend in a direction having a radial component, the through bores being formed in a region of the socket which socket inner surface is, in the assembled state, in contact with the ball outer surface, and which socket comprises plugs designed to be inserted in the through bores to form protrusions protruding from the socket inner surface and contacting the ball outer surface to increase friction between the ball and the socket when the conduit is under vacuum, to lock the relative position of the plurality of segments and of the plurality of connectors. For this purpose, the conduit is connected to means to apply vacuum. In this embodiment, the envelope is designed to form an airtight volume with a valve at a proximal end of the steerable device to allow a connection with a vacuum pump. In the relaxed state, i.e. a state in which no vacuum is applied to the device tip, the plugs inserted in the through bores allow a free movement of the ball in the socket. As a result of vacuum application, the plugs are sucked into the through bores until they contact the ball outer surface thereby increasing friction between ball and socket.

Preferably, the through bores extend radially to simplify the production.

Preferably, the through bores, preferably at least three through bores, are arranged in a plane parallel to the equatorial plane of the socket inner surface at an equal peripheral distance from each other to allow for a symmetrical repartition of forces acting on the ball. Stiffness of the locked state can be dimensioned in function of the number of through bores and plugs used.

In a preferred embodiment, ball-joint connections of the plurality of ball-joint connections have each time a ball and a socket comprising torque transmission means in the form of a boss protruding radially outwards and of a recess in which the boss is inserted in the assembled state, the boss and the recess being designed to cooperate with each other to allow the transmission of torque between the ball and the socket, wherein the ball comprises the boss protruding radially outwards and the socket comprises the recess or the ball comprises the recess and the socket comprises the boss protruding radially inwards.

Preferably, in addition, a shape of the boss and a shape of the recess as well as a relative position of the boss and the recess are dimensioned such that the recess and the boss remain always at least partially engaged when the segment and the connector move relative to each other. This feature avoids that torque transmission is lost for example when the inclination angle is too acute. Indeed, an acute inclination angle results in an increased distance between the boss and the recess, specifically in a region of the ball-joint connection opposed to the acute inclination angle.

In a preferred embodiment, the boss is a pin, preferably cylindrical, projecting radially and the recess is an axial slit, preferably in the form of an elongated rectangle having rounded angles, having a width dimensioned to allow play between the pin and edges of the recess in a peripheral direction.

In a preferred embodiment, the recess, preferably in the form of the slit, is closed on the side facing the ball-joint connection to avoid that the boss, preferably in the form of the pin, slides out of the recess. The arrangement provides for an improved security during navigation while allowing torque transmission.

In a preferred embodiment, a plurality of bosses and a plurality of recesses are arranged each time at an equal peripheral distance from each other as seen each time in a plane perpendicular to the device longitudinal axis. The symmetry resulting from this arrangement simplifies the navigation of the device.

In a preferred embodiment, the boss has the form of an elongated projection in the form of a finger running axially at the inner socket surface or at the outer ball surface and the recess is formed as an axial slit in the ball or the socket, respectively. The recess has a shape complementary to the elongated projection, preferably in the form of an elongated rectangle, having a width dimensioned to allow play between the projection and the recess in a peripheral direction.

Further, a length of the recess and a length of the elongated projection are dimensioned such that the recess and the elongated projection remain at least partially engaged when the segment and the connector move relative to each other. This feature avoids that torque transmission is lost for example when the inclination angle is too acute.

In a preferred embodiment, ball-joint connections, preferably all ball-joint connections for the sake of simplifying the production, of the plurality of ball-joint connections have a ball which ball outer surface has a structured area and a socket which socket inner surface has a further structured area to increase friction between the ball and the socket in particular when the plurality of segments and the plurality of connectors are in a locked state. This arrangement increases the efficacy of locking in the locked state because friction between the ball outer surface and the socket inner surface blocks further the relative movement of the segments and of the connectors.

### DESCRIPTION OF THE FIGURES

FIG. 1 shows a schematic perspective of a distal portion of a steerable device according to a first embodiment of the invention;

FIG. 2 shows a schematic perspective of a portion of a device tip according to a second embodiment of the invention;

FIG. 3 shows a cross section of the portion of the device tip according to the embodiment of FIG. 2;

FIG. 4 shows a schematic perspective of a portion of a device tip according to a third embodiment of the invention;

FIG. 5 shows a cross section of the portion of the device tip according to the embodiment of FIG. 4;

FIG. 6 shows a cross section of a portion of a device tip according to a fourth embodiment of the invention;

FIG. 7 shows a schematic perspective of a portion of a device tip according to a fifth embodiment of the invention;

FIG. 8 shows a cross section of the portion of the device tip according to the embodiment of FIG. 7 with an envelope and granular material;

FIG. 9 shows a cross section of a ball-joint connection for use in a device tip according to the invention;

FIG. 10 shows a schematic perspective of the ball-joint connection according to the embodiment of FIG. 9;

FIG. 11 shows a schematic perspective of a ball-joint connection for use in a device tip according to the invention;

FIG. 12 shows a cross section of the ball-joint connection according to the embodiment of FIG. 11;

FIG. 13 shows a cross section view of a ball-joint connection for use in a device tip according to the invention;

FIG. 14 shows a schematic perspective of a ball-joint connection for use in a device tip according to the invention;

FIG. 15 shows a cross section of the ball-joint connection according to the embodiment of FIG. 14;

FIG. 16 shows a schematic perspective of a ball-joint connection for use in a device tip according to the invention;

FIG. 17 shows a cross section of the ball-joint connection according to the embodiment of FIG. 16; and

FIG. 18 shows a schematic perspective of a distal portion of a steerable device according to a sixth embodiment of the invention without envelope.

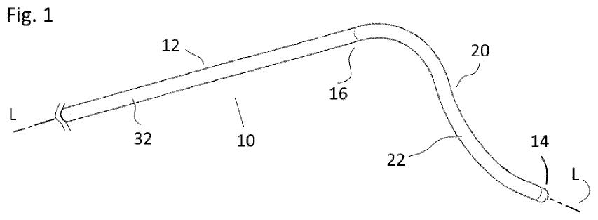

A first embodiment of the steerable device 10 according to the invention is configured as a catheter as illustrated in FIG. 1.

The steerable device 10 has an at least approximately rotationally symmetrical structure in relation to its longitudinal axis with a narrow circular section in proportion to its length. It comprises a flexible elongated element 12 in the form of an elongate shaft, whose longitudinal axis corresponds to the longitudinal axis L of the steerable device 10, having a circular section and extending over the length of the steerable device 10. The steerable device 10 has a proximal end that is not represented in FIG. 1 and a distal end 14 on the side of an operation site.

Further, the steerable device 10 comprises a flexible device tip 20 arranged at a distal end 16 of the elongated element 12. The device tip 20 is configured to be bent by way of an external magnetic field to allow steering of the elongated element. In particular, the elongated element 12 ends distally in the device tip 20. The device tip 20 is bent under the action of a magnetic field in a deformed state.

The device tip 20 comprises a tubular section 22 and a plurality of connectors 24 as well as plurality of segments 26.

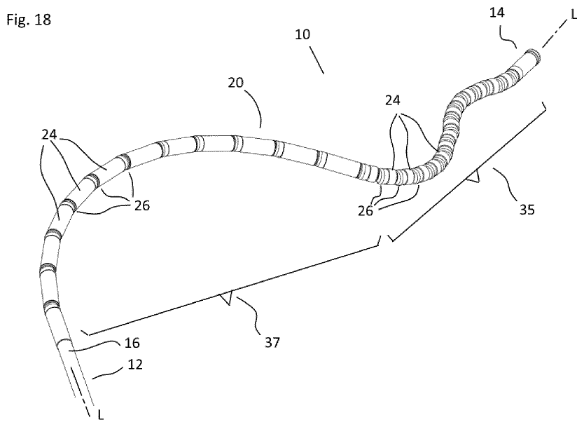

The tubular section 22 is formed as a tube enveloping the plurality of connectors 24 as well as plurality of segments 26 and extending distally in the longitudinal direction from the elongated element 12, flush with an external envelope 32 of the elongated element. The embodiment represented in FIG. 18 has a same general construction as the embodiment of FIG. 1 but is represented without the external envelope 32 and the tubular section 22.

The tubular section 22 keeps the segments 24 and the connectors 26 aligned in the steerable device 10 in the longitudinal direction L. Further, each connector connects two consecutive segments in a manner allowing movements of the segments and the connectors relative to each other in an assembled state thereof, the connection being in the form of ball-joint connections between segments and connectors.

In the embodiment of FIG. 18, the device tip 20 comprises a first group 35 of segments and connectors arranged distally, which segments and connectors have magnetic elements 34 and have a first length, as measured along the device longitudinal axis. The device tip 20 also comprises a second group 37 of segments and connectors arranged proximally, which segments and connectors are free of magnetic elements and have a second length, as measured along the device longitudinal axis L, the first length being shorter than the second length.

Consequently, there are two regions in the device tip 20, namely a distal region of the device tip including the first group 35 with magnetic elements allowing navigation of the distal region under the effect of magnetic fields, and a proximal region of the device tip including the second group 37 that is non responsive to magnetic fields but has more axial stiffness.

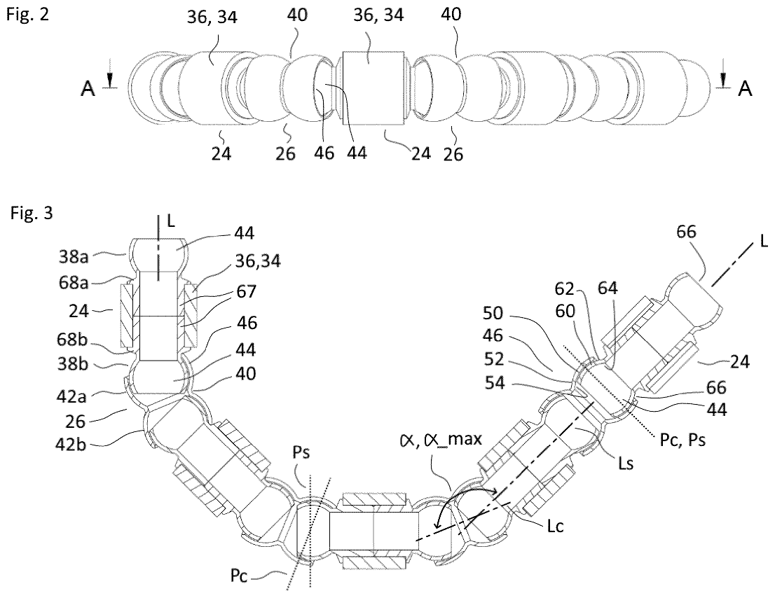

The portion of a device tip according to a second embodiment disclosed in FIG. 2 and in FIG. 3 as viewed in the cross-section A-A of FIG. 2 comprises a plurality of segments 24, each segment including a segment body 36 and a segment interface 38a and 38b arranged at each longitudinal end of the segment body 38. A segment 24 has each time two segment interfaces, namely a distal segment interface 38a and a proximal segment interface 38b, wherein the distal and proximal orientation have been arbitrarily chosen and represented in FIG. 2 and FIG. 3 as well as in the following figures.

Additionally, the device tip comprises a plurality of connectors 26, each connector including a connector body 40 and a connector interface 42a and 42b arranged at each longitudinal end of the connector body 40. A connector 26 has each time two connector interfaces, namely a distal connector interface 42a and a proximal connector interface 42b.

The plurality of segments and the plurality of connectors are arranged along the device longitudinal axis L, each connector 26 connecting two consecutive segments 24 in a manner allowing movements of the segments and the connectors relative to each other in an assembled state thereof to allow bending of the device tip.

The plurality of segments 24 and the plurality of connectors 26 can be arranged in a tubular section which is not represented in FIG. 2 and FIG. 3 as well as in the following figures except for FIG. 8.

The connector interfaces 42a and 42b and the segment interfaces 38a and 38b have each time mutually complementing shapes allowing rotational movements relative to each other and forming a connection, the connection being each time in the form of a ball-joint connection, to retain the connectors 26 and the segments 24 aligned along the longitudinal axis in a mated condition. Presently, the segments 24 and the connectors 26 have an axial symmetry.

The ball-joint connection comprises each time a ball 44 and a socket 46 arranged to connect a segment interface 38a and 38b to an adjacent connector interface 42b and 42a, respectively, in the direction of the device longitudinal axis L. An inclination angle α is formed each time between a segment longitudinal axis Ls of a segment and a connector longitudinal axis Lc of a connector adjacent to the segment when the device tip is bent. The inclination angle α is measured in a plane formed by the segment longitudinal axis Ls and the connector longitudinal axis Lc.

The ball-joint connections form a plurality of ball-joint connections. The balls 44 and the sockets 46 form each time together interlocking components of a snap-fit assembly forming the ball-joint connections.

In the embodiment disclosed, the ball-joint connection is formed in a manner such that the segment interfaces 38a and 38b comprise each time the ball 44 and the connector interfaces 42a and 42b comprise each time the socket 46 designed to receive the ball 44.

The sockets 46 have each time a socket wall 50 that is delimited by a socket outer surface 52 and a socket inner surface 54. The balls 44 are hollow and have each time a ball wall 60 that is delimited by the ball outer surface 62 and a ball inner surface 64.

Each ball-joint connection is formed such that the ball outer surface 52 is spherical and the socket inner surface 64 is spherical. The ball 44 extends axially further, as seen in the direction from ball to socket, than an equatorial plane of the socket Ps, the socket 46 having an opening 66 with an opening diameter that is less than a ball diameter of the ball, an edge region of the socket comprising the opening forming a detent provided to retain the ball and the socket in a mated condition. The opening of the socket lies preferably in a plane that is parallel to the equatorial plane of the socket and has a circular form.

For the sake of completeness, it is noted that the equatorial plane Ps of the socket 46 extends perpendicular to the segment longitudinal axis Ls and comprises the center of the sphere defined by the socket inner surface 54. Similarly, the equatorial plane Pb of the ball 44 extends perpendicular to the connector longitudinal axis Lc and comprises the center of the sphere defined by the ball outer surface 64.

The connectors 26 are made up of one piece, wherein the connector bodies 40 are limited to a narrow section connecting each time the connector interfaces, here specifically the sockets. The segments 24 are made up of individual components each time in the form of the segment body 36 formed as a tube element, and the segment interfaces 38a and 38b, wherein the segment interfaces 38a and 38b are inserted in the segment body 36 at each end of the segment body.

For an easy insertion in the segment bodies 36, the segment interfaces 38a and 38b have an insertion portion 67 for guiding the insertion, the insertion portion 67 being arranged on their side facing the segment body 36.

Each segment 24 and each connector 26 have a bore 65 extending in the direction of the device longitudinal axis L and forming a plurality of bores, the plurality of bores forming a conduit 69 extending axially through the plurality of segments, i.e. through the segment bodies and the segment interfaces, and through the plurality of connectors, i.e. through the connector bodies and the connector interfaces, in the assembled state.

Magnetic elements 34 are associated to segment bodies 36. Presently, the magnetic elements 34 are associated to the segment bodies in the form of magnetic tubes forming the segment bodies themselves, i.e. in which the segment interface 38a and 38b are inserted.

The segments interfaces 38a and 38b have each time a collar 68a and 68b extending radially outwards and designed to form an abutment limiting the insertion of the segment interfaces 38a and 38b in the segment body 36 on its distal side and on its proximal side when the segment is assembled.

At the same time, the collar is dimensioned and designed on its side facing the connector interface to form a contact position on which the edge region of the socket can abut when the device tip is bent, and a maximal inclination angle α_max is reached.

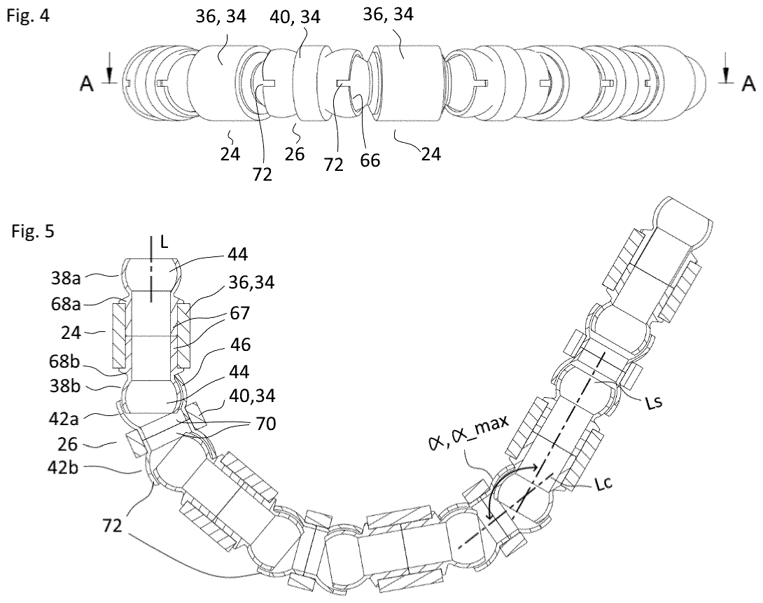

The portion of a device tip according to a third embodiment disclosed in FIG. 4 and in FIG. 5 as viewed in the cross-section A-A of FIG. 4 has a general construction like the one of the second embodiment. For the sake of conciseness, only differences are discussed and same parts or parts having the same function are referred to with same reference number as for the second embodiment.

The segments 24 of the third embodiment are the same as those of the second embodiment. In contrast to the second embodiment, the connectors 26 are made up of individual components each time in the form of the connector body 40 formed as a tube element, and the connector interfaces 42a and 42b, wherein the connector interfaces 42a and 42b are inserted in the connector body 40 at each end of the segment body.

For an easy insertion in the connector bodies 40, the connector interfaces 42a and 42b have an insertion portion 70 for guiding the insertion, the insertion portion 70 being arranged on their side facing the connector body 40.

For an easier assembly of the ball-joint connections, the connector interfaces 42a and 42b, specifically the sockets, have at least one cut 72 extending axially from the opening 66 of the socket in the direction opposed to the opening. The cuts allow an easier insertion of the balls in the sockets because they provide for expansion of the socket.

In addition to the magnetic elements 34 associated to segment the bodies 36 in the form of magnetic tubes forming the segment bodies themselves, magnetic elements 34 are also associated to the connector bodies, also in the form of magnetic tubes forming the connector bodies themselves, i.e. in which each time the connector interfaces 42a and 42b are inserted. This embodiment allows the increase of magnetic material present in the device tip.

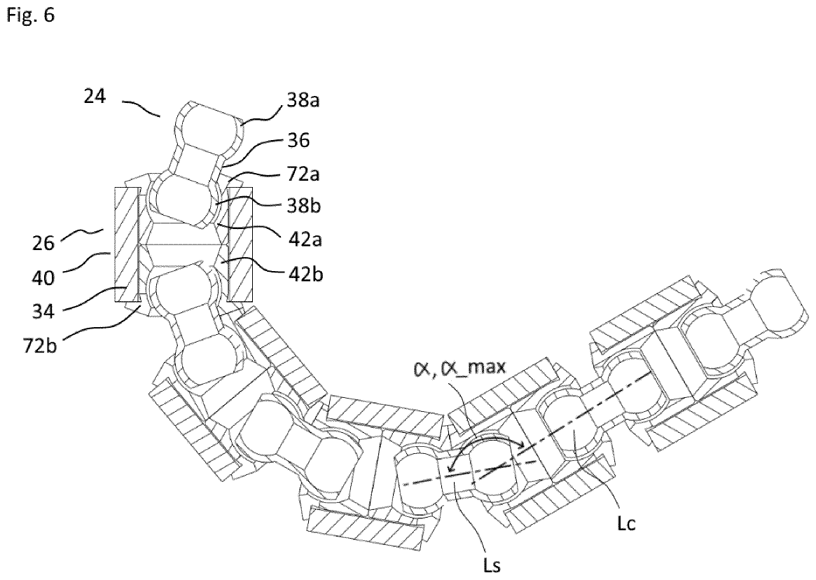

The fourth embodiment illustrated in FIG. 6 has an inverse construction of the second embodiment, specifically regarding the assembly of the segments and connectors as well as the arrangement of the magnetic elements.

The segments 24 are made up of one piece, wherein the segment bodies 36 are limited to a narrow section connecting each time the connector interfaces 38a and 38b, here specifically the sockets. The connectors 26 are made up of individual components each time in the form of the connector body 40 formed as a tube element, and the connector interfaces 42a and 42b, wherein the connector interfaces 42a and 42b are inserted in the connector body 40 at each end of the connector body.

The connector interfaces 42a and 42b have each time a collar 72a and 72b extending radially outwards and designed to form an abutment limiting the insertion of the connector interfaces 42a and 42b in the connector body 40 on its distal side and on its proximal side when the connector is assembled.

At the same time, the collar is dimensioned and designed on its side facing the following connector to form a contact position on which the collar of the following connector can abut when the device tip is bent, and a maximal inclination angle α max is reached. This embodiment allows the formation of a robust abutment between the two collars.

In contrast to the second embodiment, the segments 24 are free of magnetic elements 34 but magnetic elements 34 are associated to the connector bodies 40, also in the form of magnetic tubes forming the connector bodies themselves, i.e. in which each time the connector interfaces 42a and 42b are inserted.

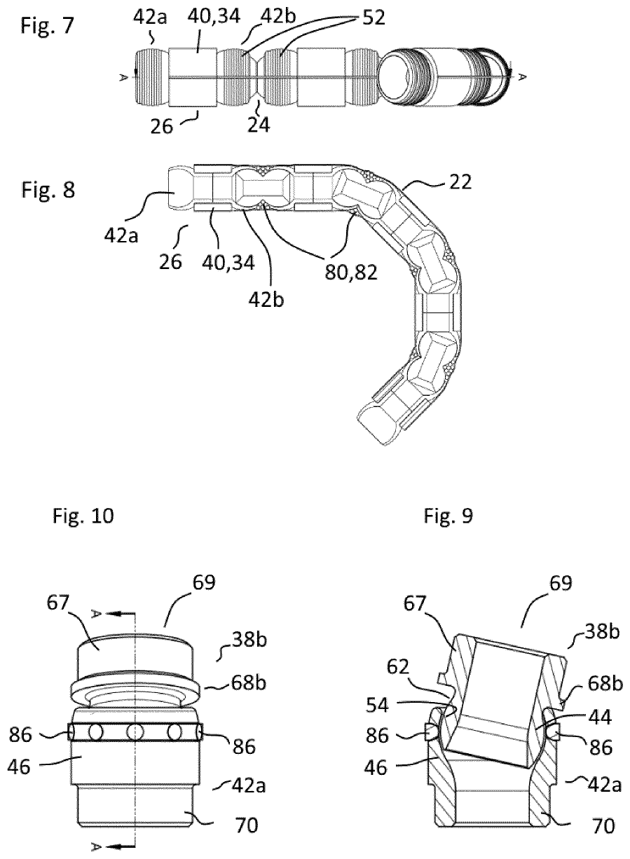

The fifth embodiment illustrated in FIG. 7 and in FIG. 8 as viewed in the cross-section A-A of FIG. 7 is different from the previous embodiments mainly in that it includes a locking mechanism of the position of the segments and the connectors relative to each other. This locking arrangement can also be used with the embodiments previously described.

In the fifth embodiment, the socket outer surfaces 52 have a structured region. The structured region has patterns in the form of a plurality of concentric grooves, spaced from each other, as seen in the longitudinal direction, each groove extending circumferentially in a plane parallel to the equatorial plane of the socket.

Further, an interspace 80 is formed, in the assembled state, by gaps between the sockets 42a and 42b and the tubular section 22, wherein the interspace 80 is filled with granular material 82. The tubular section 22 surrounds the plurality of segments 24 and the plurality of connectors 26 and is designed to form an airtight volume with a vacuum source (not represented) for example by way of a valve at a proximal end of the steerable device to allow a connection with a vacuum pump.

In this embodiment, when the interspace is under vacuum, the tubular section is pressed against the plurality of segments and the plurality of connectors as well as against granular material provided each time between segments and connectors. As a result, friction between granular material itself and between granular material and the sockets blocks the relative movement of the segments and of the connectors.

FIG. 10 shows a schematic perspective of a ball-joint connection also represented in FIG. 9 as viewed in the cross-section A-A of FIG. 10. The ball-joint connection includes a locking mechanism of the position of the segments and the connectors relative to each other. The arrangement can also be used with the embodiments previously described.

The socket 46 of the ball-joint connection comprises through bores 84 that extend radially and have a taper extending inwards. The through bores 84 are arranged in a plane parallel to the equatorial plane of the socket inner surface at an equal peripheral distance from each other to allow for a symmetrical repartition of forces acting on the ball 44. The through bores 84 are formed in a region of the socket which socket inner surface 54 is, in the assembled state, in contact with the ball outer surface 62.

Further, the socket 46 comprises plugs 86 designed to be inserted in the through bores 84, wherein the taper of the through bores is each time designed to block the plugs in the through bores to allow the formation of protrusions protruding from the socket inner surface 54.

In an embodiment of the device tip in which the ball-joint connection is used, the tubular section 22 surrounding the plurality of segments 24 and the plurality of connectors 26 is designed to form an airtight volume with a vacuum source In a relaxed state, i.e. a state in which no vacuum is applied to the device tip, the plugs 86 inserted in the through bores 84 allow a free movement of the ball in the socket. As a result of vacuum application, for example by way of a vacuum pump, the conduit 69 is under vacuum and the plugs 86 are sucked into the through bores 84 until they are blocked in the through bores and contact the ball outer surface, thereby increasing friction between ball and socket. As a result, the relative position of the plurality of segments and of the plurality of connectors is locked.

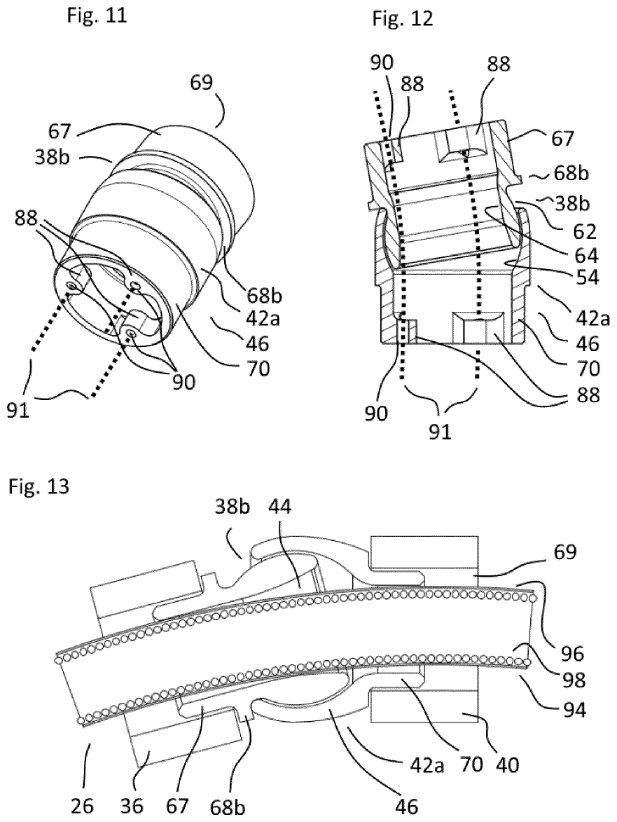

FIG. 11 shows a schematic perspective of a ball-joint connection also represented in FIG. 12 as viewed in the cross-section A-A of FIG. 11. The ball-joint connection includes a locking mechanism of the position of the segments and the connectors relative to each other. The arrangement can also be used with the embodiments previously described.

The segments and the connectors comprise each time at least three passages 88 in the form of lugs arranged in the bores forming the conduit 69 and projecting radially inwards in the conduit. The lugs are arranged at an equal peripheral distance from each other in a plane perpendicular to the segment longitudinal axis and to the connector longitudinal axis, respectively, to allow for a symmetrical application of forces.

The at least three passages 88 have each a through hole 90. In the assembled state, the through holes 90 of the segments and the through holes of the connectors are aligned and form at least three guides through the plurality of segments and the plurality of connectors. The at least three guides are designed to allow each time the passage of a pull wire 91 schematically represented in FIG. 11 and FIG. 12 that is fixed in a distal region of the device tip. The pull wires 91 are designed to be pulled proximally to lock the relative position of the plurality of segments and the plurality of connectors by increasing friction each time between the ball outer surface 62 on the socket inner surface 54.

In the present embodiment, the lugs are arranged in the segment interface 38b and in the connector interface 42a. As a result, each guide wire 91 is supported in each segment and each connector by two lugs.

FIG. 13 shows a cross-section of a ball-joint connection including a locking mechanism of the position of the segments and the connectors relative to each other. The arrangement can also be used as an alternative to the use of pull wires previously described.

In the present embodiment, a sleeve 94 is arranged in the conduit 69, the sleeve comprising a braided sheath 96 and a spring element 98 surrounded by the braided sheath 96. In a relaxed state of the sleeve 94, the sleeve has an external diameter equal to or smaller than an internal diameter of the conduit 69 and the spring element 98 has an external diameter equal to or smaller than an inner diameter of the braided sheath 96. The sleeve extends from a distal sleeve end (not represented) fixed in a distal region of the device tip to a proximal sleeve end (not represented). The proximal sleeve end is designed to be pulled proximally to switch from the relaxed state to a stretched state of the sleeve 94 in which the relative position of the plurality of segments 24 and of the plurality of connectors 26 is locked. To prevent the braided sheath 96 to collapse and/or shrink in diameter while an operator pulls the sleeve, the spring element, which can be a wire spring, is designed to maintain the inner diameter of the braided sheath constant or at least approximately constant. A distal end of the spring element is fixed in the distal region of the device tip at the same position as the distal sleeve end. The distal sleeve end can be fixed for example by gluing or mechanically. In an embodiment comprising ball-joint connections, the distal sleeve end can be fixed mechanically for example at the most distal ball or if the case may be the most distal joint of the arrangement formed by the plurality of segments and the plurality of connectors. In an embodiment comprising a most distal segment made of individual components in the form of a segment body and segment interfaces configured to be inserted in the segment body, the distal sleeve end can be arranged in a manner as to protrude from the most distal segment, wherein the distal sleeve end is fixed by pinching it between the distal segment interface and the segment body in the mounted state of the segment. For this purpose, the distal sleeve end can be extended radially outwards, folded in the proximal direction and pulled over the segment interface in the proximal direction to envelop the outer surface of the segment interface so that, in the mounted state of the segment, at least a portion of the distal sleeve end is pinched between the distal segment interface and the segment body. The same arrangement is also conceivable in an embodiment comprising a most distal connector made of individual components in the form of a connector body and connector interfaces.

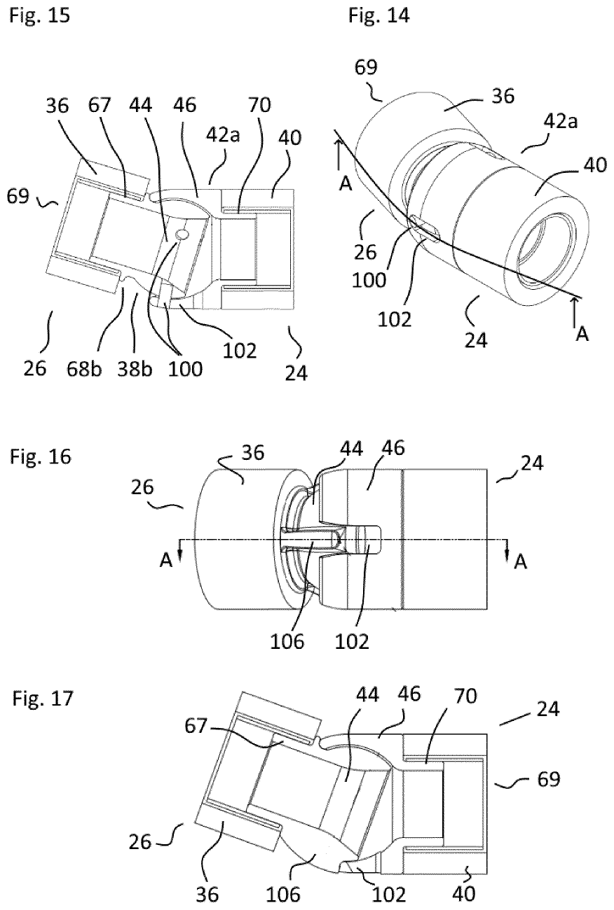

FIG. 14 shows a schematic perspective of a ball-joint connection also represented in FIG. 15 as viewed in the cross-section A-A of FIG. 14. The ball-joint connection includes torque transmission means to transmit torque from segments to connectors along the device tip. The arrangement can also be used with the embodiments previously described.

In the present embodiment, the ball-joint connection has a ball 44 and a socket 46 comprising torque transmission means in the form of a cylindrical pin 100 protruding radially outwards from the ball 44 and of a recess 102. The recess 102 is an axial slit in the form of an elongated rectangle having rounded angles, the rectangle having a width dimensioned to form a play between the pin and edges of the recess in a peripheral direction. In the present embodiment, the play allows only a sliding contact of the pin in the recess 102. It is also noted that the recess 102 is closed on the side facing the ball-joint connection to avoid that the pin 100 slides out of the recess.

Embodiments in which the recess 102 is open on the side facing the ball-joint connection are also possible. However, in this case, the relative position of the pin 100 and the recess 102 are dimensioned such that the recess 102 and the pin 100 remain always at least partially engaged when the segment 26 and the connector 24 move relative to each other. This feature avoids that torque transmission is lost for example when the inclination angle α is too acute.

In the assembled state, the pin 100 is inserted in the recess, wherein the pin 100 and the recess 102 are designed to cooperate with each other to allow the transmission of torque between the ball and the socket.

In the embodiment disclosed, three pairs comprising a pin and a recess engaged with each other are provided and arranged each time at an equal peripheral distance from each other, as seen each time in a plane perpendicular to the device longitudinal axis, i.e. as seen in a plane perpendicular to the segment longitudinal axis for the ball and to the connector longitudinal axis for the socket.

FIG. 16 shows a schematic side view of a ball-joint connection also represented in FIG. 17 as viewed in the cross-section A-A of FIG. 16. The ball-joint connection includes also torque transmission means to transmit torque from segments to connectors along the device tip. The arrangement can also be used with the embodiments previously described.

In the present embodiment, the ball-joint connection has a ball 44 and a socket 46 comprising torque transmission means including a finger 106 running at the inner surface of the ball and the recess 102.

The recess 102 is formed as an axial slit in the socket 46 in the form of an elongated rectangle having rounded angles, the rectangle having a width dimensioned to allow play between the finger 106 and edges of the recess in a peripheral direction. The recess 102 is open on the side facing the ball-joint connection to allow the introduction of the finger 106 in the recess 102 in the assembled state. A length of the recess 102 and a length of the finger 106 are dimensioned such that the recess and the elongated projection remain at least partially engaged when the segment 26 and the connector 24 move relative to each other, in particular when they reach the maximal inclination angle α_max.

In the embodiment disclosed, three pairs comprising a finger and a recess engaged with each other are provided and are arranged each time at an equal peripheral distance from each other, as seen each time in a plane perpendicular to the device longitudinal axis, i.e. as seen in a plane perpendicular to the segment longitudinal axis for the ball and to the connector longitudinal axis for the socket.

List of reference numbers

| | |
|---|---|
| steerable device | 10 |
| elongated element | 12 |
| distal end of the steerable device | 14 |
| distal end of the elongated element | 16 |
| device tip | 20 |
| tubular section | 22 |
| connectors | 24 |
| segments | 26 |
| external envelope of elongated element | 32 |
| magnetic elements | 34 |
| segment body | 36 |
| distal and a proximal segment interface | 38a and 38b |
| connector body | 40 |
| distal and a proximal connector interface | 42a, 42b |
| ball | 44 |
| socket | 46 |
| socket wall | 50 |
| socket outer surface | 52 |
| socket inner surface | 54 |
| ball wall | 60 |
| ball outer surface | 62 |
| ball inner surface | 64 |
| bore | 65 |
| opening of the socket | 66 |
| insertion portion | 67 |
| collar of segment interface | 68a and 68b |
| conduit | 69 |
| insertion portion of connector interface | 70 |
| collar of connector interface | 72a and 72b |
| interspace | 80 |
| granular material | 82 |
| through bores | 84 |
| plugs | 86 |
| passages | 88 |
| through holes | 90 |
| pull wire | 91 |
| sleeve | 94 |
| braided sheath | 96 |
| spring element | 98 |
| pin | 100 |
| recess | 102 |
| finger | 106 |
| device longitudinal axis | L |
| segment longitudinal axis | LS |
| connector longitudinal axis | Lc |

1. Steerable device for use inside of a mammalian body, comprising:
an elongated element elongated along a device longitudinal axis and configured to be navigated through the mammalian body;
the elongated element having a device tip which is flexible and configured for allowing steering of the elongated element by way of an external magnetic field,
the device tip comprising a plurality of segments, each segment including a segment body and a segment interface arranged at each longitudinal end of the segment body, and a plurality of connectors, each connector including a connector body and a connector interface arranged at each longitudinal end of the connector body, arranged along the device longitudinal axis, each connector connecting two consecutive segments in a manner allowing movements of the segments and the connectors relative to each other in an assembled state thereof to allow bending of the device tip;
wherein
the connector interfaces and the segment interfaces have each time mutually complementing shapes allowing rotational movements relative to each other and forming a connection to retain the connectors and the segments aligned along the device longitudinal axis in a mated condition, and in that magnetic elements form at least partially segment bodies or connector bodies or both.

2. Steerable device according to claim 1, wherein the magnetic elements each time surround at least partially the segment bodies or the connector bodies or both, or are at least partially embedded in the segment bodies or in the connector bodies or in both.

3. Steerable device according to claim 1, wherein the connectors are made up of individual components in the form of the connector body, and of the connector interfaces, the latter being inserted in the connector body at each end of the connector body, and/or the segments are made up of individual components in the form of the segment body, and of the segment interfaces, interfaces, the latter being inserted in the segment body at each end of the segment body.

4. Steerable device according to claim 3, wherein the tube elements or the further tube elements or both form the magnetic elements.

5. Steerable device according to claim 1, wherein the device tip comprises a first group of segments and connectors, which segments and connectors have a first length and a second group of segments and connectors, which segments and connectors have a second length, as measured along the device longitudinal axis, the first length being shorter than the second length.

6. Steerable device according to claim 1, wherein the device tip comprises a first group of segments and connectors, which segments and connectors have magnetic elements and a second group of segments and connectors, which segments and connectors are free from magnetic elements.

7. Steerable device according to claim 1, wherein the device tip comprises segments having different lengths and connectors having different lengths.

8. Steerable device according to claim 1, wherein the connection is a ball-joint connection comprising each time a ball and a socket arranged to connect a segment interface to an adjacent connector interface in the direction of the device longitudinal axis, the socket having a socket inner surface and a socket outer surface and the ball having a ball outer surface, an inclination angle α being formed when the device tip is bent each time between a segment longitudinal axis of a segment and a connector longitudinal axis of a connector adjacent to the segment, the ball-joint connections forming a plurality of ball-joint connections.

9. Steerable device according to claim 8, wherein the ball of ball-joint connections sits each time on a neck extending axially, and in that, for the segment and the connector of the ball-joint connections, the inclination angle α is limited to a maximal inclination angle α_max reached when the neck is in a contact position with an edge of the socket of the ball-joint connection as a result of a relative movement of the segment with respect to the connector, the edge acting as a stopper for the inclination of the segment relative to the connector.

10. Steerable device according to claim 8, wherein the ball of ball-joint connections is truncated on the side of the ball facing the socket by a first plane, forming a ball abutment surface and the socket of the plurality of ball-joint connections having a planar socket bottom, on its side facing the ball, and in that, for the segment and the connector of each ball-joint connection of the plurality of ball-joint connections, the inclination angle α is limited to a maximal inclination angle α_max reached when the ball abutment surface is in a contact position with the socket bottom as a result of a relative movement of the segment with respect to the connector of the ball-joint connection, the contact position acting as a stopper for the inclination of the segment relative to the connector.

11. Steerable device according to claim 8, wherein the socket outer surfaces have a structured region, and in that an interspace formed in the assembled state between the sockets and an envelope of the steerable device is filled with granular material, the envelope surrounding at least the plurality of segments and the plurality of connectors, wherein the granular material locks the relative position of the plurality of segments and of the plurality of connectors when the interspace is under vacuum, the interspace being connected to means to apply vacuum.

12. Steerable device according to claim 8, wherein each segment and each connector have a bore extending in the direction of the device longitudinal axis and forming a plurality of bores, the plurality of bores forming a conduit through the plurality of segments and the plurality of connectors in the assembled state.

13. Steerable device according to claim 12, wherein the segments and the connectors include each time at least three passages arranged in the respective bores forming the conduit, the at least three passages forming each a through hole, wherein, in the assembled state, the through holes of the segments and the through holes of the connectors are aligned and the through holes form at least three guides through the plurality of segments and the plurality of connectors, the at least three guides being designed to allow each time the passage of a pull wire, wherein the pull wire is fixed in a distal region of the device tip and is designed to be pulled proximally to lock the relative position of the plurality of segments and the plurality of connectors.

14. Steerable device according to claim 12, wherein a sleeve is arranged in the conduit, the sleeve comprising a braided sheath and a spring element surrounded by the braided sheath, wherein, in a relaxed state of the sleeve, the sleeve has an external diameter equal to or smaller than an internal diameter of the conduit and the spring element has an external diameter equal to or smaller than an inner diameter of the braided sheath, the sleeve extending from a distal sleeve end fixed in a distal region of the device tip to a proximal sleeve end designed to be pulled proximally to switch from the relaxed state to a stretched state of the sleeve in which the relative position of the plurality of segments and of the plurality of connectors is locked.

15. Steerable device according to claim 12, wherein ball-joint connections which socket comprises through bores extending in a direction having a radial component, the through bores having an inwards taper and being formed in a region of the socket which socket inner surface is, in the assembled state, in contact with a ball outer surface of the ball, and which socket comprises plugs designed to be introduced in the through bores to form protrusions protruding from the socket inner surface and contacting the ball outer surface to increase friction between the ball and the socket when the conduit is under vacuum, to lock the relative position of the plurality of segments and of the plurality of connectors.

16. Steerable device according to claim 8, wherein ball-joint connections which ball and socket have torque transmission means in the form of a boss protruding radially outwards and of a recess in which the boss is inserted in the assembled state, the boss and the recess being designed to cooperate with each other to allow the transmission of torque between the ball and the socket, wherein the ball comprises the boss protruding radially outwards and the socket comprises the recess or the ball comprises the recess and the socket comprises the boss protruding radially inwards.

17. Steerable device according to claim 16, wherein the boss is in the form of a pin projecting radially and the recess is in the form of an axial slit having a width dimensioned to form a play between the pin and edges of the recess in a peripheral direction.

18. Steerable device according to claim 8, wherein ball-joint connections of the plurality of ball-joint connections have a ball which outer surface has a structured area and a socket which inner surface has a further structured area to increase friction between the ball and the socket.
